(19) United States
(12) Patent Application Publication
(10) Pub. No.: US 2016/0175296 A1
(43) Pub. Date: Jun. 23, 2016

(54) Method of producing uniform buprenorphine-containing formulations

(71) Applicant: ARx, LLC, Glen Rock, PA (US)

(72) Inventors: William Curtis Baer, II, Harrisburg, PA (US); Michael Joseph Passariello, York, PA (US)

(21) Appl. No.: 14/757,886

(22) Filed: Dec. 23, 2015

Related U.S. Application Data
(60) Provisional application No. 62/096,212, filed on Dec. 23, 2014.

(51) Int. Cl. A61K 31/485; A61K 9/70

(52) U.S. Cl. CPC A61K 31/485; A61K 9/7015

(57) ABSTRACT

A method of forming a liquid formulation includes preparing a mixture and adding a second component to the mixture to form the liquid formulation. The mixture includes water, a film-forming polymer, a buffer, and a first component. The first component is an active ingredient including buprenorphine or a sweetener. The second component is the active ingredient comprising buprenorphine or the sweetener. Adding the second component is the last step in creating the liquid formulation. If the first component is the active ingredient, the second component is the sweetener. If the first component is the sweetener, the second component is the active ingredient. A method of forming a water-disintegrable film includes casting a formulation film from the liquid formulation and drying the formulation film to form the water-disintegrable film. A film formulation and a water-disintegrable film are also disclosed.

### REFERENCE TO RELATED APPLICATIONS

This application claims one or more inventions disclosed in U.S. Provisional Patent Application No. 62/096,212, filed Dec. 23, 2014, entitled “METHOD OF PRODUCING UNIFORM BUPRENORPHINE-CONTAINING FORMULATIONS”. The benefit under 35 USC §119(e) of the United States provisional application is hereby claimed, and the aforementioned application is hereby incorporated herein by reference.

### FIELD OF THE INVENTION

The present application is directed toward the field of pharmaceutical formulations and more particularly to uniform buprenorphine-containing formulations and methods of producing uniform buprenorphine-containing formulations used for drug delivery.

### BACKGROUND OF THE INVENTION

Immediate-release pharmaceutical formulations are well-known and come in various forms, such as syrups, lozenges, thin films, transmucosal patches, sublingual tablets, orally-disintegrating tablets, nasal sprays, metered dose inhalers, and sublingual films to name a few examples. Fast-dissolving drug-delivery systems were first developed in the late 1970s as an alternative to tablets, capsules, and syrups for pediatric and geriatric patients who experience difficulties swallowing traditional oral solid-dosage forms. In response to this need, a variety of orally disintegrating tablet (ODT) formats were commercialized. Most ODT products were formulated to dissolve in less than one minute when exposed to saliva to form a solution that could then be more easily swallowed.

More recently, dissolvable oral thin films (OTFs) emerged from the confection and oral care markets in the form of breath strips. These products became a widely accepted form by consumers for delivering vitamins and personal care products and subsequently for also delivering other active ingredients, including pharmaceuticals.

Pharmaceutical companies and consumers alike have embraced OTFs as a practical and accepted alternative to traditional medicine forms, such as liquids, tablets, and capsules. OTFs offer fast, accurate dosing in a safe, efficacious format that is convenient and portable, without the need for water or measuring devices. OTFs are typically no larger than the size of a postage stamp and disintegrate on or under a patient's tongue in a matter of seconds for the rapid release of one or more active pharmaceutical ingredients (APIs). More broadly, the use of thin films has expanded to include a variety of products that are manufactured and used for a wide range of transmucosal drug delivery applications beyond oral GI delivery.

The recent social movement towards opioid abuse has created a market for drug products that are designed to curb the withdrawal symptoms associated with opioid addiction. Pharmaceutical products have been marketed for the treatment of opioid addiction. These drug products have been available to prescribers and patients as an oral tablet, as a transmucosal delivery device, and as a sublingual film dosage form, and they contain the active ingredient buprenorphine.

Sublingual dosage forms disintegrate in the oral cavity, typically under the tongue. Transmucosal delivery devices erode in the oral cavity while affixed to the buccal side of the cheek. As such, importance is placed on the sensory perception of the dosage form so that the patient experiences no displeasure or bad taste that might cause one to discontinue use of this important medication for treating their withdrawal symptoms. Most oral film products and sublingual tablet formulations contain flavors and sweeteners that mask bitterness or off notes associated with the active ingredients contained within the dosage forms. These flavors and sweeteners are accepted pharmaceutical excipients that meet certain pharmaceutical or food compendia monographs.

Despite the various buprenorphine delivery modes via films, syrups, tablets, metered dose inhalers, or nasal sprays, there still exists opportunities for improvements to such products when formulated with selected sweeteners, and there are a variety of commercial needs in the field that have not yet been met.

### BRIEF DESCRIPTION OF THE INVENTION

In an embodiment, a method of forming a liquid formulation includes preparing a mixture and adding a second component to the mixture to form the liquid formulation. The mixture includes water, a film-forming polymer, a buffer, and a first component. The first component is an active ingredient including buprenorphine or a sweetener. The second component is the active ingredient including buprenorphine or the sweetener. Adding the second component is the last step in creating the liquid formulation. If the first component is the active ingredient, the second component is the sweetener. If the first component is the sweetener, the second component is the active ingredient.

In another embodiment, a method of forming a water-disintegrable film includes preparing a mixture, adding a second component to the mixture to form a liquid formulation, casting a formulation film from the liquid formulation, and drying the formulation film to form the water-disintegrable film. The mixture includes water, a film-forming polymer, a buffer, and a first component. The first component is an active ingredient including buprenorphine or a sweetener. The second component is the active ingredient including buprenorphine or the sweetener. Adding the second component is the last step in creating the liquid formulation. If the first component is the active ingredient, the second component is the sweetener. If the first component is the sweetener, the second component is the active ingredient.

In another embodiment, a liquid formulation includes a water-disintegrable film-forming polymer, buprenorphine, and sucralose, neotame, or a combination thereof.

In another embodiment, a water-disintegrable film includes a film-forming polymer, buprenorphine, and a sweetener. The sweetener includes sucralose, neotame, or a combination thereof. The buprenorphine is uniformly distributed in the water-disintegrable film.

### DETAILED DESCRIPTION OF THE INVENTION

Exemplary embodiments are directed to liquid formulations of buprenorphine, including, but not limited to, oral syrups, sublingual sprays, and liquid formulations of buprenorphine that are employed as an intermediate to form a solid, such as films for oral or transmucosal drug delivery, including, but not limited to, dissolvable oral thin films, sublingual thin films, and transmucosal patches, and the production of the same, including those that address currently existing but unmet needs. More particularly, exemplary embodiments are directed to unit dose forms of those thin films.

The composition of the films discussed in the context of exemplary embodiments may be characterized broadly as a liquid-base biologically compatible film-forming polymer matrix containing buprenorphine that forms a water-soluble film upon drying and may include, without limitation, those described in U.S. Pat. No. 7,470,397, which is hereby incorporated by reference herein in its entirety. It should be appreciated that the resulting films have a combination of a solid content sufficient to provide film strength to aid in handling but balanced to provide disintegration at a predetermined rate.

It should also be appreciated that any liquid formulations, discussed herein and that are intended to be used as an intermediate for forming a dried film, may also be used as an oral syrup or as a sublingual spray simply by adjusting the viscosity of the formulation prior to packaging or dispensing. Typically the amount of solvent or combination of solvents is adjusted in such a way that provides the optimum viscosity for dispensing the dosage form.

In some embodiments, a specific order of addition to form the liquid formulation is provided to minimize an unavoidable precipitation of buprenorphine during mixing with a less desirable sweetener. In some embodiments, the less desirable sweetener is Acesulfame potassium, sodium saccharin, or a combination thereof.

Any suitable polymers may be employed as the matrix of the thin film in accordance with exemplary embodiments. It should be appreciated that the polymer(s) selected for any particular film may depend on a variety of factors, including the active ingredient to be incorporated, the desired rate of disintegration (which may be modified with or without the use of a surfactant), and the viscosity of the liquid formulation used to form the films, as well as other factors known to those of ordinary skill in the art for producing conventional thin films.

The polymer may be water-soluble, water-swellable, water-insoluble, or a combination thereof and may include cellulose or a cellulose derivative. Although the use of water-swellable and water-insoluble polymers is contemplated, the formulation preferably contains a sufficient amount of water-soluble polymer to ensure the eventual disintegration of the subsequently formed film.

Exemplary polymers for the film-forming matrix include, but are not limited to, water-soluble hydroxypropylmethyl cellulose (HPMC), hydroxyethyl cellulose, hydroxypropyl cellulose, polyvinyl pyrrolidone, carboxymethyl cellulose, sodium carboxymethyl cellulose, methyl cellulose, polyvinyl alcohol, sodium alginate, polyethylene glycol/polyethylene oxide, xanthan gum, tragacantha, guar gum, acacia gum, arabic gum, carrageenan, polyacrylic acid, methylmethacrylate copolymer, carboxyvinyl copolymers, various mixtures of the above, or other known water-soluble polymers, cellulose derivatives, or gums. Other polymers that may be used include, but are not limited to, ethyl cellulose, hydroxypropyl ethyl cellulose, cellulose acetate phthalate, hydroxypropyl methyl cellulose phthalate, or combinations thereof.

In some embodiments, the polymer matrix may include a surfactant to adjust the rate of dissolution. In other embodiments, the rate of dissolution may be adjusted by the use of a combination of high and low molecular weight polymers with or without the use of a surfactant. For example, particularly beneficial properties of film strength and disintegration profile (i.e. the rate at which the film disintegrates upon contact with the oral cavity or other mucosa) are obtained when the water-soluble components include a combination of low molecular weight polymers (e.g., those having a molecular weight less than about 5,000 to about 60,000 daltons) and high molecular weight polymers (e.g., those having a molecular weight of about 60,000 to about 150,000 daltons, up to about 900,000 daltons, or higher).

Additional water-soluble polymers include, but are not limited to, polyvinyl alcohol-polyethylene glycol copolymer, such as Kollicoat® IR by BASF SE (Ludwigshafen, Germany), which has a molecular weight of about 49,000 daltons and a sodium salt of an acrylic polymer, such as Acrysol by Rohm and Haas (Philadelphia, Pa.), which is available in various grades having different molecular weights.

Various other polymers may be selected by one of ordinary skill in the art given the teachings herein and preferably include a sufficient amount of a high molecular weight component to impart adequate film strength and a sufficient amount of a low molecular weight component to facilitate the desired film property of the disintegration profile.

According to another exemplary embodiment of the invention, the water-soluble low molecular weight component need not be a water-soluble polymer. Instead, the low molecular weight component may be a low molecular weight monomer or a combination of various low molecular weight monomers. The low molecular weight component serves to promote disintegration but is present in an amount such that film strength is adequate for processing and dispensing. Various concentrations of the low molecular weight component may be utilized.

The amounts of high and low molecular weight components may be adjusted to achieve a desired, predetermined disintegration profile for the film, which may range from a few seconds to several minutes or even hours. When slower disintegration is desired, the concentration of the high molecular weight component is preferably increased relative to the concentration of the low molecular weight component. When faster disintegration is desired, the concentration of the low molecular weight component is preferably increased relative to the concentration of the high molecular weight component. Additionally, the thickness of the film may be adjusted to achieve a desired disintegration profile. To increase the disintegration time, the film thickness is increased. To decrease the disintegration time, the film thickness is decreased. Adequate film strength should be maintained, however, to allow for handling of the film.

In addition to buprenorphine, polymers, and sweeteners, other ingredients that may be incorporated into the film formulation include, but are not limited to, a plasticizer, starch, thickener, buffer, stabilizer, flavorings, other additives, and combinations thereof, which are preferably, but not necessarily, water-soluble. The types and amounts of such ingredients are familiar to those within the art for formulating conventional water-soluble thin films.

Films in accordance with exemplary embodiments also include buprenorphine and one or more active ingredients, typically a pharmaceutical drug. A wide range of active ingredients in addition to buprenorphine may be incorporated into the liquid formulation prior to film formation. These active ingredients may be incorporated in any form using a liquid carrier, including as a solution, emulsion, suspension, or dispersion. The specific form may depend upon the particular combination of active ingredient and polymer to be employed. That is, active-containing liquid formulations that are used to create the films may be in the form of a solution in which all ingredients, including drug substances, are fully dissolved and soluble in the bulk liquid; as an emulsion, typically used for aqueous formulations to which an oil-soluble ingredient such as a flavoring has been added; and suspensions or dispersions in which insoluble active ingredients or other excipients may be added to the bulk-liquid formulation while still achieving uniformity of distribution in the subsequently formed film.

Active ingredients that may be included in the film along with buprenorphine include, by way of example and not of limitation, ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-Parkinson agents, anti-rheumatic agents, appetite stimulants, biological response modifiers, blood modifiers, bone metabolism regulators, cardiovascular agents, central nervous system stimulants, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, or combinations thereof. The types and amounts of active ingredients to be employed are familiar to those within the art for formulating conventional dissolvable thin films.

Known methods of film production involve casting the intermediate liquid formulation onto a continuous substrate (e.g. paper or polyester liners which may or may not have release coatings) to form wide, long rolls or what are sometimes referred to as master rolls. The manufacturing process includes drying the liquid formulation to remove water and other solvents to yield the thin film on the substrate. The master rolls thus formed are then converted into smaller unit doses through a combination of roll slitting and individual unit dose die-cutting, as well as transferring those doses from the manufacturing substrate to the primary product packaging.

The sweetener or sweeteners selected for any particular film formulation may depend on a variety of factors, including the active ingredient to be incorporated, the desired level of sweetening effect (which may be modified by concentration of selected sweetener), the order of addition for the excipients and active ingredient, as well as other factors known to those of ordinary skill in the art for producing conventional film formulations. However, certain sweeteners have been found to cause premature buprenorphine precipitation. Surprisingly, it has been found that sweeteners which cause buprenorphine precipitation may be employed if a particular order of addition is followed during formulating the liquid intermediate. The particular order of addition requires that the sweetener be added as the final ingredient or that the buprenorphine be added as the final ingredient.

Sweeteners that may be included in the formulation include, by way of example and not of limitation, Acesulfame potassium, advantame, aspartame, corn sugar, dextrose, erythritol, fructose, galactose, glycerol, high fructose corn syrup, high maltose corn syrup, isomalt, lactitol, lactose, maltitol, maltodextrin, maltose, mannitol, neotame, saccharin, sucrose, sorbitol, sucralose, tagatose, trehalose, and xylitol.

Accordingly, exemplary embodiments overcome buprenorphine solubility issues through careful selection of sweeteners that maintain buprenorphine solubility in the liquid blend in a more desired manner. Maintaining higher levels of buprenorphine solubility in the liquid blend helps ensure that a relatively consistent amount of buprenorphine is present in each dose as an even distribution of the active ingredient in the polymer matrix is more readily controlled in the liquid form. In particular, exemplary embodiments enable the use of certain sweeteners that have a tendency to precipitate buprenorphine, such as Acesulfame potassium (aka Acesulfame K) and sodium saccharin. Other sweeteners, such as sucralose, sorbitol, erythritol, and neotame, may also be used, and surprisingly, may result in greater buprenorphine solubility regardless of order of addition.

In some embodiments, a sweetener and a concentration of the sweetener are selected such that the sweetener does not cause premature buprenorphine precipitation during preparation of a water-disintegrable film including buprenorphine and the sweetener.

In some embodiments, a concentration of sweetener and an order of addition of components of a water-disintegrable film are selected such that the sweetener does not cause premature buprenorphine precipitation during preparation of a water-disintegrable film including buprenorphine and the sweetener. In some embodiments, the order of addition of components includes adding the buprenorphine as the last component to form the formulation from which the water-disintegrable film is formed. In other embodiments, the order of addition of components includes adding the sweetener as the last component to form the formulation from which the water-disintegrable film is formed.

In some embodiments, a sweetener and a concentration of the sweetener are selected such that the sweetener does not cause a non-uniform distribution of buprenorphine in a water-disintegrable film including buprenorphine and the sweetener.

In some embodiments, a concentration of sweetener and an order of addition of components of a water-disintegrable film are selected such that the sweetener does not cause a non-uniform distribution of buprenorphine in a water-disintegrable film including buprenorphine and the sweetener. In some embodiments, the order of addition of components includes adding the buprenorphine as the last component to form the formulation from which the water-disintegrable film is formed. In other embodiments, the order of addition of components includes adding the sweetener as the last component to form the formulation from which the water-disintegrable film is formed.

Among other advantages, the use of a particular sweetener limits variation of the buprenorphine between dosage units that may occur throughout the dry film coating during conventional master roll formation.

According to some exemplary embodiments, a method for formulating the liquid blend to be used to form a film in a unit dose form is to add the sweetener as the final ingredient. According to other exemplary embodiments, a method for formulating the liquid blend to be used to form a film in a unit dose form is to add the buprenorphine as the final ingredient. Both methods are successful in achieving a uniform liquid blend process, insofar as the ingredient that provides adequate viscosity to the liquid is fully hydrated and thereby may provide suspending aid to the buprenorphine precipitate to assure adequate uniformity throughout the mixture.

In the case of coating thin films, the liquid film-forming formulation typically has a high solids content with a moderate amount of liquid carrier and typically has the consistency of a thick syrup. The liquid is generally a thixotropic fluid with a predetermined viscosity and rheology. It will be appreciated that the characteristics of a particular liquid may depend upon the constituents in the formulation. Generally, the viscosity is in the range of about 2 KcP to about 30 KcP. Shear rates may vary, but are typically in the range of about 1 s⁻¹ to about 10 s⁻¹.

After a thin coat of the liquid has been deposited onto the coating substrate, the liquid carrier in the formulation is driven off by any suitable method to yield a dissolvable thin film. Exemplary drying methods include exposure to ambient air, infra-red (IR) heating, forced air and/or hot-air systems, and combinations thereof.

After the film on the substrate is dried, the web is rolled up to form a master roll. Further processing yields unit dose films that are individually formed by die-cutting and sealed into individual packages, each package containing a single unit dose film.

The invention is further described by way of the following examples, which are presented by way of illustration, not of limitation.

### EXAMPLE 1

To determine percent solubility of buprenorphine in a 30% solids formulation, the following ingredients were added sequentially as listed in Table 1 and stirred to form a uniform mixture.

TABLE 1
Example 1 Composition

| Component | Amount (g) | % |
|---|---|---|
| Water | 68.989 | 68.99 |
| Maltitol Syrup | 4.070 | 4.07 |
| FD&C Yellow #6 | 0.009 | 0.01 |
| Lime Flavor | 0.643 | 0.64 |
| Citric Acid, anhydrous | 2.536 | 2.54 |
| Trisodium Citrate, anhydrous | 1.148 | 1.15 |
| Acesulfame Potassium | 0.643 | 0.64 |
| HPMC | 1.808 | 1.81 |
| Polyethylene Oxide | 17.780 | 17.78 |
| Naloxone HCl, dihydrate | 0.523 | 0.52 |
| Buprenorphine HCl | 1.851 | 1.85 |
| Total | 100.00 | 100.00 |

Results: The mixture was pulled through a 0.45 μm polytetrafluoroethylene (PTFE) filter in order to extract the soluble portion of buprenorphine. The filtered solution was coated directly onto a substrate using a knife coating apparatus. The gap thickness was 0.026″. Once the filtered solution was deposited onto the substrate, it was dried using an electric-forced air oven at 65° C. for 40 minutes to yield a film with mass of 70 mg and an area 5.63 cm². The film was placed into a 50 mL volumetric flask. 40 mL of diluent was added. The contents were sonicated for 10 minutes. 4 mL of methanol was added. The contents were sonicated for an additional 10 minutes. 6 mL of solution was then pipetted into a 50 mL volumetric flask, and diluted to volume with diluent. Buprenorphine assay was determined to be 21.39% by reverse phase High Performance Liquid Chromatography (HPLC). By reference, buprenorphine HCl reportedly has an aqueous solubility of 17 mg/mL.

### EXAMPLE 2

Buprenorphine particle size distribution was measured on the liquid from Example 1 by dissolving the liquid in a saturated buprenorphine aqueous solution and collecting the test results on a Malvern Mastersizer® 2000S (Malvern Instruments Ltd., Malvern, Worcestershire, England). The distribution test result for the Example 1 liquid was d[0.1]=3.647μ, d[0.5]=8.632μ, d[4,3]=14.718μ, d[0.9]=22.803μ.

### EXAMPLE 3

To determine percent solubility of buprenorphine in a 25% solids formulation, the 30% solids mixture from Example 1 was diluted with water as shown in Table 2 and stirred to form a theoretical 25% solids mixture.

TABLE 2
Example 3 Composition

| Component | Amount (g) | % |
|---|---|---|
| Mixture from Example 1 | 39.437 | 83.33 |
| Water | 7.887 | 16.67 |
| Total | 47.324 | 100.00 |

Results: Example 3 was prepared similarly to that which is described in Example 1 and submitted for HPLC analysis. Buprenorphine assay was determined to be 20.65%.

### EXAMPLE 4

Buprenorphine particle size distribution was measured on the liquid from Example 3 by dissolving the liquid in a saturated buprenorphine aqueous solution and collecting the test results on a Malvern Mastersizer® 2000S. The distribution test result for Example 3 liquid was d[0.1]=4.394μ, d[0.5]=9.608μ, d[4,3]=12.342μ, d[0.9]=22.479μ.

### EXAMPLE 5

To determine percent solubility of buprenorphine in a 30% solids formulation that does not contain polymer and naloxone HCl, the following ingredients were added sequentially as listed in Table 3 and stirred to form a uniform mixture.

TABLE 3
Example 5 Composition

| Component | Amount (g) | % |
|---|---|---|
| Water | 23.00 | 86.35 |
| Maltitol Syrup | 1.360 | 5.11 |
| FD&C Yellow #6 | 0.003 | 0.01 |
| Natural Key Lime Flavor | 0.214 | 0.80 |
| Citric Acid, anhydrous | 0.845 | 3.17 |
| Trisodium Citrate, anhydrous | 0.383 | 1.44 |
| Acesulfame Potassium | 0.214 | 0.80 |
| Buprenorphine HCl | 0.617 | 2.32 |
| Total | 26.64 | 100.00 |

Results: The mixture was pulled through a 0.45 μm PTFE filter in order to extract the soluble portion of buprenorphine. 1 mL of the filtered solution was pipetted into a 100 mL volumetric flask. 8 mL of methanol was added, followed by 80 mL of diluent. The contents were then sonicated for 10 minutes. 2 mL of solution was then pipetted into a 50 mL volumetric flask, and diluted to volume with diluent. Buprenorphine soluble fraction was determined to be 0.680% by reverse phase HPLC.

### EXAMPLE 6

To determine percent solubility of buprenorphine in the presence of citric acid/trisodium citrate, the following ingredients were added sequentially as listed in Table 4 and stirred to form a uniform mixture. The ingredient concentrations represent those that would be present in a 30% theoretical solids formulation that contained all formulation components. This formulation was prepared to test the buprenorphine solubility in the buffering system.

TABLE 4
Example 6 Composition

| Component | Amount (g) | % |
|---|---|---|
| Water | 23.00 | 92.57 |
| Citric Acid, anhydrous | 0.845 | 3.40 |
| Trisodium Citrate, | 0.383 | 1.54 |
| anhydrous | | |
| Buprenorphine HCl | 0.617 | 2.48 |
| Total | 24.85 | 100.00 |

Results: Example 6 was prepared similarly to that which is described in Example 5 and submitted for HPLC analysis. Buprenorphine soluble fraction, in the buffer concentrations from a representative formulation at 30% theoretical solids, was determined to be 83.2%.

### EXAMPLE 7

To determine percent solubility of buprenorphine in the presence of citric acid/trisodium citrate, the following ingredients were added sequentially as listed in Table 5 and stirred to form a uniform mixture. The ingredient concentrations represent those that would be present in a 25% theoretical solids formulation that contained all formulation components. This formulation was prepared to test the buprenorphine solubility in the buffering system targeted for pH=4.

TABLE 5
Example 7 Composition

| Component | Amount (g) | % |
|---|---|---|
| Water | 25.00 | 94.23 |
| Citric Acid, anhydrous | 0.700 | 2.64 |
| Trisodium Citrate, | 0.320 | 1.21 |
| anhydrous | | |
| Buprenorphine HCl | 0.510 | 1.92 |
| Total | 26.530 | 100.00 |

Results: Example 7 was prepared similarly to that which is described in Example 5 and submitted for HPLC analysis. Buprenorphine soluble fraction, in the buffer concentrations from a representative formulation at 25% theoretical solids, was determined to be 96.6%.

### EXAMPLE 8

To determine percent solubility of buprenorphine after various ingredient additions, the following ingredients were added sequentially as listed in Table 6 and stirred to form a uniform mixture. The ingredient concentrations represent those that would be present in a 25% theoretical solids formulation that contained all formulation components.

TABLE 6
Example 8 Compositions

| | Component | Amount (g) | % |
|---|---|---|---|
| Step 1 | Water | 72.764 | 90.13 |
| | Citric Acid, anhydrous | 2.078 | 2.57 |
| | Trisodium Citrate, anhydrous | 0.936 | 1.16 |
| | Buprenorphine HCl | 1.513 | 1.87 |
| | Maltitol Syrup | 3.441 | 4.26 |
| | Total | 80.732 | 100.00 |
| Step 2 | Water | 71.412 | 90.12 |
| | Citric Acid, anhydrous | 2.039 | 2.57 |
| | Trisodium Citrate, anhydrous | 0.919 | 1.16 |
| | Buprenorphine HCl | 1.485 | 1.87 |
| | Maltitol Syrup | 3.377 | 4.26 |
| | FD&C Yellow #6 | 0.0078 | 0.01 |
| | Total | 79.240 | 100.00 |
| Step 3 | Water | 70.060 | 89.50 |
| | Citric Acid, anhydrous | 2.000 | 2.55 |
| | Trisodium Citrate, anhydrous | 0.902 | 1.15 |
| | Buprenorphine HCl | 1.457 | 1.86 |
| | Maltitol Syrup | 3.313 | 4.23 |
| | FD&C Yellow #6 | 0.008 | 0.01 |
| | Lime Flavor | 0.539 | 0.69 |
| | Total | 78.279 | 100.00 |
| Step 4 | Water | 69.717 | 89.02 |
| | Citric Acid, anhydrous | 1.962 | 2.51 |
| | Trisodium Citrate, anhydrous | 0.885 | 1.13 |
| | Buprenorphine HCl | 1.429 | 1.82 |
| | Maltitol Syrup | 3.250 | 4.15 |
| | FD&C Yellow #6 | 0.008 | 0.01 |
| | Lime Flavor | 0.529 | 0.68 |
| | Acesulfame Potassium | 0.532 | 0.68 |
| | Total | 78.312 | 100.00 |

Results: Samples from steps 1, 2, 3, and 4 of Example 8 were prepared similarly to that which is described in Example 5 and submitted for HPLC analysis. Buprenorphine soluble fraction was determined to be 91.764%, 90.578%, 91.678%, and 1.556% after steps 1, 2, 3, and 4, respectively. The soluble fraction testing data indicates that Acesulfame potassium significantly decreases the soluble fraction of buprenorphine.

### EXAMPLE 9

To determine percent solubility of buprenorphine, where buprenorphine HCl is added after Acesulfame potassium, the following ingredients were added sequentially as listed in Table 9 and stirred to form a uniform mixture. The mixture was formulated at 25% theoretical solids.

TABLE 7
Example 9 Composition

| Component | Amount (g) | % |
|---|---|---|
| Water | 74.176 | 75.20 |
| Maltitol Syrup | 3.472 | 3.52 |
| FD&C Yellow #6 | 0.0075 | 0.01 |
| Lime Flavor | 0.536 | 0.54 |
| Citric Acid, anhydrous | 2.113 | 2.14 |
| Trisodium Citrate, anhydrous | 0.960 | 0.97 |
| Acesulfame Potassium | 0.535 | 0.54 |
| Buprenorphine HCl | 1.569 | 1.59 |
| Naloxone HCl, dihydrate | 0.439 | 0.45 |
| Polyethylene Oxide | 14.835 | 15.04 |
| Total | 98.643 | 100.00 |

Results: Example 9 sample was prepared similarly to that which was described in Example 5 and submitted for HPLC analysis. In this Example, neither buprenorphine nor Acesulfame potassium was the last component to be added. Buprenorphine soluble fraction was determined to be 1.410% prior to the addition of polyethylene oxide polymer. The addition of Acesulfame potassium, prior to the addition of buprenorphine HCl, inhibits buprenorphine solubility.

### EXAMPLE 10

Buprenorphine particle size distribution was measured on the formulation prepared in Example 9 by dispersing the resulting liquid in a saturated buprenorphine aqueous solution and collecting the test results on a Malvern Mastersizer® 2000S. The distribution test result for Example 9 formulation was d[0.1]=17.364μ, d[0.5]=66.039μ, d[4,3]=79.488μ, d[0.9]=162.772μ. Since the resultant particle size is essentially equivalent to the particle size distribution of the starting buprenorphine, it was concluded that Acesulfame potassium inhibits buprenorphine solubility.

### EXAMPLE 11

To determine the percent solubility of buprenorphine, where buprenorphine HCl is added before Acesulfame potassium, the following ingredients were added sequentially as listed in Table 8 and stirred to form a uniform mixture. The mixture was formulated at 25% theoretical solids.

TABLE 8
Example 11 Composition

| Component | Amount (g) | % |
|---|---|---|
| Water | 74.125 | 74.13 |
| Maltitol Syrup | 3.438 | 3.44 |
| FD&C Yellow #6 | 0.0079 | 0.01 |
| Lime Flavor | 0.538 | 0.54 |
| Citric Acid, anhydrous | 2.113 | 2.11 |
| Trisodium Citrate, anhydrous | 0.959 | 0.96 |
| HPMC | 1.517 | 1.52 |
| Polyethylene Oxide | 14.825 | 14.83 |
| Naloxone HCl, dihydrate | 0.437 | 0.44 |
| Buprenorphine HCl | 1.546 | 1.55 |
| Acesulfame Potassium | 0.490 | 0.49 |
| Total | 99.996 | 100.00 |

Results: Example 11 sample was prepared similarly to that which was described in Example 1 and submitted for HPLC analysis. Buprenorphine soluble fraction was determined to be 94.828% and 22.012%, before and after Acesulfame potassium addition, respectively. The addition of Acesulfame potassium significantly decreased the solubility of buprenorphine in the formulation, as indicated by precipitation of previously-dissolved buprenorphine. The composition of Example 11 is similar to the composition of Example 9, but in Example lithe Acesulfame potassium was added last, which resulted in a significantly higher fraction of soluble buprenorphine than in Example 9.

### EXAMPLE 12

Buprenorphine particle size distribution was measured on the formulation prepared in Example 11 prior to and after the addition of Acesulfame potassium by dispersing the resulting liquids in a saturated buprenorphine aqueous solution and collecting the test results on a Malvern Mastersizer® 2000S. The distribution test results for Example 11 formulations were d[0.1]1.899μ, d[0.5]=5.914μ, d[4,3]=7.318μ, d[0.9]=47.481μ before Acesulfame potassium addition and d[0.1]=5.331μ, d[0.5]=16.135μ, d[4,3]=25.648μ, d[0.9]=60.492μ after Acesulfame potassium addition. This shows that for Example 11, the addition of Acesulfame potassium caused the buprenorphine particle size to become larger due to reduced solubility.

### EXAMPLE 13

To determine percent solubility of buprenorphine, where Acesulfame potassium is substituted with sodium saccharin dihydrate as an alternative sweetener, the following ingredients were added sequentially as listed in Table 9 and stirred to form a uniform mixture. The mixture was formulated at 25% theoretical solids.

TABLE 9
Example 13 Composition

| Component | Amount (g) | % |
|---|---|---|
| Water | 74.153 | 88.58 |
| Maltitol Syrup | 3.453 | 4.12 |
| FD&C Yellow #6 | 0.0076 | 0.01 |
| Lime Flavor | 0.540 | 0.65 |
| Citric Acid, anhydrous | 2.112 | 2.52 |
| Trisodium Citrate, anhydrous | 0.970 | 1.16 |
| Naloxone HCl, dihydrate | 0.438 | 0.52 |
| Buprenorphine HCl | 1.546 | 1.85 |
| Sodium Saccharin, dihydrate | 0.493 | 0.59 |
| Total | 83.713 | 100.00 |

Results: Example 13 sample was prepared similarly to that which was described in Example 5 and submitted for HPLC analysis. Buprenorphine soluble fraction was determined to be 0.146%. Sodium saccharin dihydrate imparts a significant effect on the precipitation of buprenorphine in the formulation.

### EXAMPLE 14

Buprenorphine particle size distribution was measured on the formulation prepared in Example 13 by dispersing the resulting liquid in a saturated buprenorphine aqueous solution and collecting the test results on a Malvern Mastersizer® 2000S. The distribution test results for Example 13 formulation was d[0.1]=8.125μ, d[0.5]=30.135μ, d[4,3]=60.554μ, d[0.9]=143.492μ.

### EXAMPLE 15

To determine percent solubility of buprenorphine, where Acesulfame potassium is substituted with sucralose as an alternative sweetener, the following ingredients were added sequentially as listed in Table 10 and stirred to form a uniform mixture. The mixture was formulated at 25% theoretical solids.

TABLE 10
Example 15 Composition

| Component | Amount (g) | % |
|---|---|---|
| Water | 74.150 | 88.57 |
| Maltitol Syrup | 3.446 | 4.12 |
| FD&C Yellow #6 | 0.0079 | 0.01 |
| Lime Flavor | 0.547 | 0.65 |
| Citric Acid, anhydrous | 2.114 | 2.53 |
| Trisodium Citrate, anhydrous | 0.966 | 1.15 |
| Naloxone HCl, dihydrate | 0.439 | 0.52 |
| Buprenorphine HCl | 1.553 | 1.86 |
| Sucralose | 0.496 | 0.59 |
| Total | 83.719 | 100.00 |

Results: Example 15 sample was prepared similarly to that which was described in Example 5 and submitted for HPLC analysis. Buprenorphine soluble fraction was determined to be 49.563%. Therefore, the addition of sucralose has less of an impact on the solubility of buprenorphine than Acesulfame potassium or sodium saccharin.

### EXAMPLE 16

Buprenorphine particle size distribution was measured on the formulation prepared in Example 15 by dispersing the resulting liquid in a saturated buprenorphine aqueous solution and collecting the test results on a Malvern Mastersizer® 2000S. The distribution test results for Example 15 formulation was d[0.1]=1.975μ, d[0.5]=3.897μ, d[4,3]=6.266μ, d[0.9]=15.413μ.

### EXAMPLE 17

To determine percent solubility of buprenorphine, where Acesulfame potassium is substituted with sorbitol as an alternative sweetener, the following ingredients were added sequentially as listed in Table 11 and stirred to form a uniform mixture. The mixture was formulated at 25% theoretical solids.

TABLE 11
Example 17 Composition

| Component | Amount (g) | % |
|---|---|---|
| Water | 74.390 | 88.46 |
| Maltitol Syrup | 3.568 | 4.24 |
| FD&C Yellow #6 | 0.0084 | 0.01 |
| Lime Flavor | 0.549 | 0.65 |
| Citric Acid, anhydrous | 2.140 | 2.54 |
| Trisodium Citrate, anhydrous | 0.970 | 1.15 |
| Naloxone HCl, dihydrate | 0.437 | 0.52 |
| Buprenorphine HCl | 1.573 | 1.87 |
| Sorbitol | 0.461 | 0.55 |
| Total | 84.096 | 100.00 |

Results: Example 17 sample was prepared similarly to that which was described in Example 5 and submitted for HPLC analysis. Buprenorphine soluble fraction was determined to be 42.618%. Therefore, the addition of sorbitol has less of an impact on the solubility of buprenorphine than Acesulfame potassium or sodium saccharin.

### EXAMPLE 18

To determine percent solubility of buprenorphine, where Acesulfame potassium is substituted with erythritol as an alternative sweetener, the following ingredients were added sequentially as listed in Table 12 and stirred to form a uniform mixture. The mixture was formulated at 25% theoretical solids.

TABLE 12
Example 18 Composition

| Component | Amount (g) | % |
|---|---|---|
| Water | 74.153 | 88.66 |
| Maltitol Syrup | 3.485 | 4.17 |
| FD&C Yellow #6 | 0.0079 | 0.01 |
| Lime Flavor | 0.542 | 0.65 |
| Citric Acid, anhydrous | 2.123 | 2.54 |
| Trisodium Citrate, anhydrous | 0.981 | 1.17 |
| Naloxone HCl, dihydrate | 0.447 | 0.53 |
| Buprenorphine HCl | 1.586 | 1.90 |
| Erythritol | 0.310 | 0.37 |
| Total | 83.635 | 100.00 |

Results: Example 18 sample was prepared similarly to that which was described in Example 5 and submitted for HPLC analysis. Buprenorphine soluble fraction was determined to be 41.994%. Therefore, the addition of erythritol has less of an impact on the solubility of buprenorphine than Acesulfame potassium or sodium saccharin.

### EXAMPLE 19

To determine percent solubility of buprenorphine, where Acesulfame potassium is substituted with neotame as an alternative sweetener, the following ingredients were added sequentially as listed in Table 13 and stirred to form a uniform mixture. The mixture was formulated at 25% theoretical solids.

TABLE 13
Example 19 Composition

| Component | Amount (g) | % |
|---|---|---|
| Water | 74.163 | 88.09 |
| Maltitol Syrup | 3.529 | 4.19 |
| FD&C Yellow #6 | 0.0073 | 0.01 |
| Lime Flavor | 0.542 | 0.64 |
| Citric Acid, anhydrous | 2.213 | 2.63 |
| Trisodium Citrate, anhydrous | 0.979 | 1.16 |
| Naloxone HCl, dihydrate | 0.437 | 0.52 |
| Buprenorphine HCl | 1.577 | 1.87 |
| Neotame | 0.738 | 0.88 |
| Total | 84.185 | 100.00 |

Results: Example 19 sample was prepared similarly to that which was described in Example 5 and submitted for HPLC analysis. Buprenorphine soluble fraction was determined to be 54.006%. Therefore, the addition of neotame has less of an impact on the solubility of buprenorphine than Acesulfame potassium or sodium saccharin.

### EXAMPLE 20

To determine percent solubility of buprenorphine, where Acesulfame potassium is substituted with sodium chloride to assess the impact of sodium ion on buprenorphine solubility, the following ingredients were added sequentially as listed in Table 14 and stirred to form a uniform mixture. The mixture was formulated at 25% theoretical solids.

TABLE 14
Example 20 Composition

| Component | Amount (g) | % |
|---|---|---|
| Water | 74.148 | 88.95 |
| Maltitol Syrup | 3.502 | 4.20 |
| FD&C Yellow #6 | 0.0075 | 0.01 |
| Lime Flavor | 0.540 | 0.65 |
| Citric Acid, anhydrous | 2.122 | 2.55 |
| Trisodium Citrate, anhydrous | 0.974 | 1.17 |
| Naloxone HCl, dihydrate | 0.439 | 0.53 |
| Buprenorphine HCl | 1.543 | 1.85 |
| Sodium Chloride | 0.0796 | 0.10 |
| Total | 83.355 | 100.00 |

Results: Example 20 sample was prepared similarly to that which was described in Example 5 and submitted for HPLC analysis. Buprenorphine soluble fraction was determined to be 52.166%. Therefore, the addition of sodium chloride ion has less of an impact on the solubility of buprenorphine than Acesulfame potassium or sodium saccharin.

### EXAMPLE 21

Acesulfame potassium was substituted with neotame to prepare a formulation for dissolution testing as listed in Table 15.

TABLE 15
Example 21 Composition

| Component | Amount (g) | % |
|---|---|---|
| Water | 74.240 | 73.84 |
| Maltitol Syrup | 3.520 | 3.50 |
| FD&C Yellow #6 | 0.0083 | 0.01 |
| Lime Flavor | 0.554 | 0.55 |
| Citric Acid, anhydrous | 2.111 | 2.10 |
| Trisodium Citrate, anhydrous | 0.967 | 0.96 |
| Neotame | 0.804 | 0.80 |
| HPMC | 1.517 | 1.51 |
| Polyethylene Oxide | 14.832 | 14.75 |
| Naloxone HCl, dihydrate | 0.434 | 0.43 |
| Buprenorphine HCl | 1.558 | 1.55 |
| Total | 100.54 | 100.00 |

The solution was coated directly onto a substrate using a knife coating apparatus. The gap thickness was 0.026″. Once the filtered solution was deposited onto the substrate, it was dried using an electric-forced air oven at 65° C. for 40 minutes to yield a film with mass of 70 mg/5.63 cm².

### EXAMPLE 22

Acesulfame potassium was substituted with sodium saccharin to prepare a formulation for dissolution testing as listed in Table 16.

TABLE 16
Example 22 Composition

| Component | Amount (g) | % |
|---|---|---|
| Water | 74.218 | 74.08 |
| Maltitol Syrup | 3.485 | 3.48 |
| FD&C Yellow #6 | 0.0081 | 0.01 |
| Lime Flavor | 0.547 | 0.55 |
| Citric Acid, anhydrous | 2.125 | 2.12 |
| Trisodium Citrate, anhydrous | 0.972 | 0.97 |
| Sodium Saccharin | 0.494 | 0.49 |
| HPMC | 1.517 | 1.51 |
| Polyethylene Oxide | 14.837 | 14.81 |
| Naloxone HCl, dihydrate | 0.433 | 0.43 |
| Buprenorphine HCl | 1.552 | 1.55 |
| Total | 100.19 | 100.00 |

A film of Example 22 was prepared similarly to that which is described in Example 21.

### EXAMPLE 23

Acesulfame potassium was substituted with sucralose to prepare a formulation for dissolution testing as listed in Table 17.

TABLE 17
Example 23 Composition

| Component | Amount (g) | % |
|---|---|---|
| Water | 74.192 | 73.67 |
| Maltitol Syrup | 3.526 | 3.50 |
| FD&C Yellow #6 | 0.0083 | 0.01 |
| Lime Flavor | 0.549 | 0.55 |
| Citric Acid, anhydrous | 2.123 | 2.11 |
| Trisodium Citrate, anhydrous | 0.977 | 0.97 |
| Sucralose | 0.986 | 0.98 |
| HPMC | 1.521 | 1.51 |
| Polyethylene Oxide | 14.832 | 14.73 |
| Naloxone HCl, dihydrate | 0.439 | 0.44 |
| Buprenorphine HCl | 1.551 | 1.54 |
| Total | 100.70 | 100.00 |

A film of Example 23 was prepared similarly to that which is described in Example 21.

### EXAMPLE 24

Example 3, Example 21, Example 22, and Example 23 were submitted for dissolution testing. The dissolution method recommended in the Food and Drug Administration (FDA) Office of Generic Drugs (OGD) dissolution methods database for buprenorphine HCl/naloxone HCl sublingual films was utilized for product characterization [900 mL, acetate buffer (at pH 4.0) in United States Pharmacopeia (USP) apparatus V (with 56 mm, 40 mesh stainless steel disc) stirred at 100 rpm]. The temperature of the dissolution media was maintained at 37±0.5° C., and the buprenorphine concentration was determined using HPLC at a wavelength of 230 nm. Three replicates were evaluated per each sampling time point in accordance with the OGD Dissolution Method recommendations.

TABLE 18
Buprenorphine Release Results
Average % Buprenorphine Released (n = 3)

| Sample | 1 min | 2 min | 3 min | 5 min | 7 min | 10 min |
|---|---|---|---|---|---|---|
| Example 21 | 78 | 104 | 104 | 104 | 103 | 103 |
| Example 23 | 57 | 81 | 86 | 91 | 95 | 98 |
| Example 34 | 8 | 89 | 95 | 99 | 102 | 103 |
| Example 22 | 28 | 82 | 93 | 99 | 99 | 99 |

The dissolution data shown in Table 18 indicates that the release of buprenorphine may be altered by changing the sweetener. Of the four sweeteners tested, buprenorphine was released the quickest when neotame was the sweetener. Buprenorphine released initially more quickly when sucralose was the sweetener than when Acesulfame potassium or sodium saccharin was the sweetener, but both Acesulfame potassium and sodium saccharin formulations reached 99% buprenorphine release within 5 minutes, whereas the sucralose formulation was only at 91% buprenorphine release at 5 minutes.

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims and all other patentable subject matter contained herein.

What is claimed is:

1. A method of forming a liquid formulation, the method comprising:
preparing a mixture comprising water, a film-forming polymer, a buffer, and a first component selected from the group consisting of:
an active ingredient comprising buprenorphine; and
a sweetener; and
adding a second component to the mixture to form the liquid formulation, the second component being selected from the group consisting of:
the active ingredient comprising buprenorphine; and
the sweetener;
wherein the step of adding the second component is the last step in creating the liquid formulation;
wherein if the first component is the active ingredient, the second component is the sweetener; and
wherein if the first component is the sweetener, the second component is the active ingredient.

2. The method of claim 1, wherein the first component is the active ingredient, and the second component is the sweetener.

3. The method of claim 1, wherein the first component is the sweetener, and the second component is the active ingredient.

4. The method of claim 1, wherein the sweetener is selected from the group consisting of Acesulfame potassium, sodium saccharin, and a combination thereof.

5. The method of claim 1, wherein the sweetener is selected from the group consisting of neotame, sucralose, sorbitol, erythritol, and combinations thereof.

6. The method of claim 1, wherein the film-forming polymer is selected from the group consisting of hydroxypropylmethyl cellulose, hydroxyethyl cellulose, hydroxypropyl cellulose, polyvinyl pyrrolidone, carboxymethyl cellulose, sodium carboxymethyl cellulose, methyl cellulose, polyvinyl alcohol, sodium alginate, polyethylene glycol, xanthan gum, tragacantha, guar gum, acacia gum, arabic gum, carrageenan, polyacrylic acid, a methylmethacrylate copolymer, a carboxyvinyl copolymer, and combinations thereof.

7. The method of claim 1, wherein a first dissolved amount of buprenorphine in the liquid formulation is greater than a second dissolved amount of buprenorphine in a reference formulation having an identical composition to the liquid formulation except that the step of adding the second component is not the last step in creating the reference formulation prior to casting.

8. The method of claim 1, wherein the mixture further comprises at least one compound selected from the group consisting of a plasticizer, a starch, a thickener, a stabilizer, a flavoring, a surfactant, and combinations thereof.

9. The method of claim 1, wherein the liquid formulation serves as a basis of a final dosage form selected from the group consisting of a water-disintegrable film, an oral syrup, and a sublingual spray.

10. A method of forming a water-disintegrable film, the method comprising:
preparing a mixture comprising water, a film-forming polymer, a buffer, and a first component selected from the group consisting of:
an active ingredient comprising buprenorphine; and
a sweetener;
adding a second component to the mixture to form a liquid formulation, the second component being selected from the group consisting of:
the active ingredient comprising buprenorphine; and
the sweetener;
casting a formulation film from the liquid formulation; and
drying the formulation film to form the water-disintegrable film;
wherein the step of adding the second component is the last step in creating the liquid formulation prior to casting;
wherein if the first component is the active ingredient, the second component is the sweetener; and
wherein if the first component is the sweetener, the second component is the active ingredient.

11. The method of claim 10, wherein the first component is the active ingredient, and the second component is the sweetener.

12. The method of claim 10, wherein the first component is the sweetener, and the second component is the active ingredient.

13. A liquid formulation comprising a water-disintegrable film-forming polymer, buprenorphine, and a sweetener selected from the group consisting of sucralose, neotame, and a combination thereof.

14. The liquid formulation of claim 13, wherein the buprenorphine is uniformly distributed in the liquid formulation.

15. The liquid formulation of claim 13, wherein the water-disintegrable film-forming polymer is selected from the group consisting of hydroxypropylmethyl cellulose, hydroxyethyl cellulose, hydroxypropyl cellulose, polyvinyl pyrrolidone, carboxymethyl cellulose, sodium carboxymethyl cellulose, methyl cellulose, polyvinyl alcohol, sodium alginate, polyethylene glycol, xanthan gum, tragacantha, guar gum, acacia gum, arabic gum, carrageenan, polyacrylic acid, a methylmethacrylate copolymer, a carboxyvinyl copolymer, and combinations thereof.

16. The liquid formulation of claim 13 further comprising at least one compound selected from the group consisting of a plasticizer, a starch, a thickener, a stabilizer, a flavoring, a surfactant, and combinations thereof.

17. The liquid formulation of claim 13, wherein the liquid formulation serves as a basis of a final dosage form selected from the group consisting of a water-disintegrable film, an oral syrup, and a sublingual spray.

18. A water-disintegrable film comprising a film-forming polymer, buprenorphine, and a sweetener selected from the group consisting of sucralose, neotame, and a combination thereof, wherein the buprenorphine is uniformly distributed in the water-disintegrable film.

19. The water-disintegrable film of claim 18, wherein the water-disintegrable film-forming polymer is selected from the group consisting of hydroxypropylmethyl cellulose, hydroxyethyl cellulose, hydroxypropyl cellulose, polyvinyl pyrrolidone, carboxymethyl cellulose, sodium carboxymethyl cellulose, methyl cellulose, polyvinyl alcohol, sodium alginate, polyethylene glycol, xanthan gum, tragacantha, guar gum, acacia gum, arabic gum, carrageenan, polyacrylic acid, a methylmethacrylate copolymer, a carboxyvinyl copolymer, and combinations thereof.

20. The water-disintegrable film of claim 18 further comprising at least one compound selected from the group consisting of a plasticizer, a starch, a thickener, a stabilizer, a flavoring, a surfactant, and combinations thereof.
